(19) United States
(12) Patent Application Publication
(10) Pub. No.: US 2021/0113383 A1
(43) Pub. Date: Apr. 22, 2021

(54) LIQUID FLOW DETECTION IN TISSUE SITE DRESSINGS AND INSTILLATION METHODS

(71) Applicant: KCI Licensing, Inc., San Antonio, TX (US)

(72) Inventors: Brett L. MOORE, San Antonio, TX (US); Hillary Renee WALKER, San Antonio, TX (US)

(21) Appl. No.: 16/981,958

(22) PCT Filed: Feb. 28, 2019
(86) PCT No.: PCT/US2019/020034

§ 371 Date: Sep. 17, 2020

Related U.S. Application Data
(60) Provisional application No. 62/646,239, filed on Mar. 21, 2018.

(51) Int. Cl. A61F 13/02; A61F 13/14; A61F 13/00; A61M 1/00

(52) U.S. Cl. CPC A61F 13/0216; A61F 13/148; A61F 13/00059; A61M 1/0084; A61F 2013/00182; A61M 1/0029; A61M 1/0001; A61F 2013/00157; A61M 1/0088

(57) ABSTRACT

A dressing for treating a tissue site may include a manifold, a transparent or translucent backing layer disposed over a surface of the manifold, an adherent layer disposed on at least a margin of the backing layer, and a plurality of liquid contact indicators disposed in a spaced array between the manifold and the backing layer. A dressing kit may include the plurality of liquid contact indicators separate from the other elements of the dressing. Therapy systems may include the dressing or a dressing assembled from the dressing kit. Also, methods for monitoring fluid distribution and methods for reducing edema can include the dressing, a dressing assembled from the dressing kit, or at least a portion of the therapy system containing them.

### RELATED APPLICATION

This application claims the benefit, under 35 U.S.C. § 119(e), of the filing of U.S. Provisional Patent Application Ser. No. 62/646,239, entitled “LIQUID FLOW DETECTION IN TISSUE SITE DRESSING AND INSTILLATION METHODS,” filed Mar. 21, 2018, which is incorporated herein by reference for all purposes.

### TECHNICAL FIELD

The invention set forth in the appended claims relates generally to tissue treatment systems and more particularly, but without limitation, to dressings, kits, and systems for tissue site therapy and methods of using same including instillation.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for improved instillation flow control, such as in a negative-pressure therapy environment, are set forth in the appended claims. Illustrative embodiments are also provided to enable a person skilled in the art to make and use the claimed subject matter.

For example, in some embodiments, a dressing or therapy system containing a dressing can be used to monitor or correct instillation fluid flow among a tissue site, such as to control instillation fluid volume or distribution. The dressing can advantageously contain a plurality of liquid contact indicators in a spaced array.

More generally, a dressing for treating a tissue site may include a manifold that may advantageously be sized and configured to fit on or within a tissue site. The manifold may include or be a porous member to allow fluid instillation or fluid removal therethrough. The dressing may also include a transparent or translucent backing layer disposed over a surface of the manifold. The dressing may further include an adherent layer disposed on at least a margin of the backing layer and configured to form a seal around the tissue site. Advantageously, the dressing may contain a plurality of liquid contact indicators disposed in a spaced array between the manifold and the backing layer. In some embodiments, at least a portion of the plurality of liquid contact indicators may be adhered to the backing layer on a surface facing the manifold. Additionally or alternatively, at least a portion of the plurality of liquid contact indicators may be adhered to the surface of the manifold proximate to the backing layer. In order to facilitate liquid detection, the plurality of liquid contact indicators may be a first color when dry and may change to a second color, different from the first color, when exposed to an aqueous fluid. In such situations, the backing layer may advantageously be transparent or translucent enough to allow an observer to visually distinguish between the first and the second color through the backing layer. In some embodiments, neither the first color nor the second color is red or pink. In some embodiments, the first color comprises white, yellow, green, or blue, and the second color comprises white, yellow, green, or blue. In some embodiments, each of the plurality of liquid contact indicators has a largest dimension from about 1 mm to about 5 mm. In some embodiments, the plurality of liquid contact indicators may include two or more different sizes, e.g., at least a first size disposed directly between the backing layer and the manifold and at least a second size disposed between the backing layer and the tissue surrounding the tissue site. In some such embodiments, the first size has a largest dimension that is larger than a largest dimension of the second size.

Alternatively, other example embodiments may include a dressing kit. The dressing kit can comprise all the elements of the dressing for treating a tissue site, but with the plurality of liquid contact indicators being included separately, such as to allow a user to orient and configure them in a spaced array between the manifold and the backing layer. In some embodiments, each of the plurality of liquid contact indicators may include an adhesive layer configured to be adhered to the backing layer or to the manifold.

Alternatively, other example embodiments may include a system for treating a tissue site, such as with negative pressure. The system may include the dressing or an assembled version of the dressing kit with the plurality of liquid contact indicators disposed in a spaced array between the manifold and the backing layer. The system may include a fluid source coupled to and in fluid communication with the dressing, and a plurality of fluid delivery pathways configured to be in fluid communication with the fluid source and to enable fluid instillation to the tissue site. In some embodiments, the system may further include a fluid flow conduit, a fluid connector subsystem for fluidly coupling the fluid source to the dressing for fluid instillation, and optionally a container fluidly coupled to the fluid source and the dressing and adapted to store or to provide fluid. In some embodiments, the system may further include a negative-pressure source fluidly coupled to the dressing and configured to enable fluid removal through the dressing. In embodiments with a negative-pressure source, the system may further include a negative-pressure conduit, a negative-pressure connector subsystem for fluidly coupling the negative-pressure source to the dressing for fluid removal, and optionally a container fluidly coupled to the negative-pressure source and the dressing and adapted to collect fluid. In some embodiments, if present, the fluid flow conduit may also function as a negative-pressure conduit. In some embodiments, if present, the fluid connector subsystem may also function as a negative-pressure connector subsystem to fluidly couple the negative-pressure source to the dressing for fluid removal through the fluid delivery pathways, which therefore may also function as fluid removal pathways to enable fluid removal through the dressing.

Methods of treatment are also described herein, wherein some example embodiments include methods for monitoring fluid distribution among a tissue site. Methods for monitoring fluid distribution may include deploying on or within the tissue site a dressing disclosed herein, at least a portion of a system for treating a tissue site disclosed herein, or a dressing kit disclosed herein, provided the plurality of separately included liquid contact indicators have been disposed in a spaced array between the manifold and the backing layer. Methods for monitoring fluid distribution may further include deploying a fluid connector subsystem, deploying a sealing member to form a fluid seal over the tissue site, fluidly coupling the fluid connector subsystem to a fluid source, and activating the fluid source to allow fluid instillation to the tissue site. In some embodiments, methods for monitoring fluid distribution may include deploying a negative-pressure connector subsystem, which can be coterminous with or separate from the fluid connector subsystem, fluidly coupling the negative-pressure connector subsystem to a negative-pressure source, and activating the negative-pressure source to allow fluid removal from the tissue site. In exemplary methods for monitoring fluid distribution, the plurality of liquid contact indicators in their array may individually change from a first color when dry to a second color, different from the first color, when exposed to an aqueous fluid. Such a color change can advantageously allow an observer to visually identify through the backing layer whether fluid instillation among the tissue site is appropriately distributed or of a proper volume. In some embodiments, a portion of the plurality of liquid contact indicators disposed between the backing layer and the tissue surrounding the tissue site may allow the observer to visually identify through the backing layer whether fluid has dispersed past a boundary of the tissue site to the tissue surrounding the tissue site.

Alternatively, other example embodiments may include methods of reducing edema for a wound surrounded by tissue. Methods of reducing edema may include providing a dressing disclosed herein, at least a portion of a system for treating a tissue site disclosed herein, or a dressing kit disclosed herein, provided the plurality of separately included liquid contact indicators have been disposed in a spaced array between the manifold and the backing layer. Methods of reducing edema may also include positioning the dressing, at least a portion of the system, or the dressing kit with the spaced array of liquid contact indicators over or within the wound, such that at least a portion of the adherent layer contacts the tissue surrounding the wound.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment in accordance with this specification;

FIG. 2 is a graph illustrating additional details of example pressure control modes that may be associated with some embodiments of the therapy system of FIG. 1;

FIG. 3 is a graph illustrating additional details that may be associated with another example pressure control mode in some embodiments of the therapy system of FIG. 1;

FIG. 4 is a chart illustrating details that may be associated with an example method of operating the therapy system of FIG. 1; and

FIGS. 5A, 5B, and 5C are schematic diagrams illustrating additional details that may be associated with an example embodiment of the therapy system of FIG. 1.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

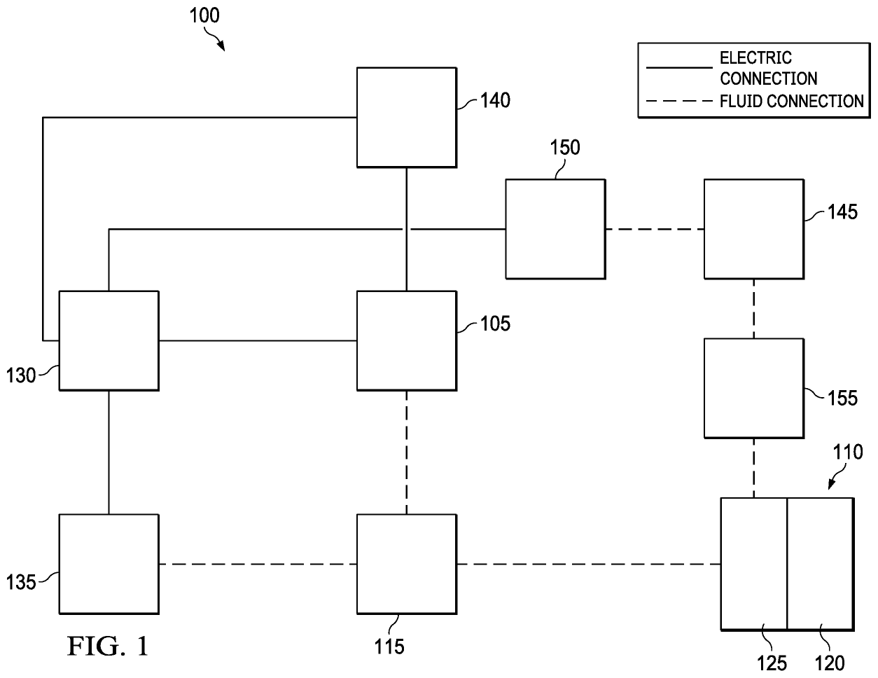

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. In some embodiments, the tissue site may include or be a peritoneal or abdominal cavity, a burn, a graft, an overhang wound, a post-operative wound, a puncture or a fistula. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Tex.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source, such as a positive-pressure source 150, a negative-pressure source such as the negative-pressure source 105, or both, in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Tex. In some embodiments, the dressing 110 containing the tissue interface 120 may include or be, for example, a V.A.C. VERAFLO CLEANSE™ dressing or a V.A.C. VERAFLO CLEANSE CHOICE™ dressing.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxyapatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and INSPIRE™ 2301 and INSPIRE™ 2327 polyurethane films, commercially available from Expopack Advanced Coatings, Wrexham, United Kingdom. In some embodiments, the cover 125 may comprise INSPIRE™ 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 145 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

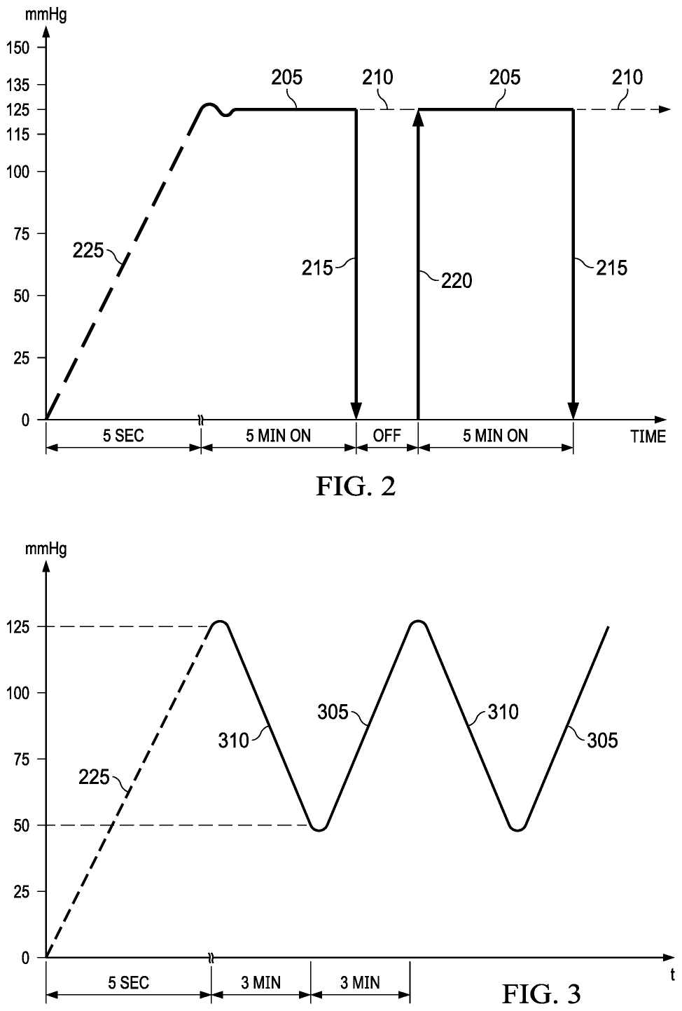

FIG. 2 is a graph illustrating additional details of an example control mode that may be associated with some embodiments of the controller 130. In some embodiments, the controller 130 may have a continuous pressure mode, in which the negative-pressure source 105 is operated to provide a constant target negative pressure, as indicated by line 205 and line 210, for the duration of treatment or until manually deactivated. Additionally or alternatively, the controller may have an intermittent pressure mode, as illustrated in the example of FIG. 2. In FIG. 2, the x-axis represents time and the y-axis represents negative pressure generated by the negative-pressure source 105 over time. In the example of FIG. 2, the controller 130 can operate the negative-pressure source 105 to cycle between a target pressure and atmospheric pressure. For example, the target pressure may be set at a value of 135 mmHg, as indicated by line 205, for a specified period of time (e.g., 5 min), followed by a specified period of time (e.g., 2 min) of deactivation, as indicated by the gap between the solid lines 215 and 220. The cycle can be repeated by activating the negative-pressure source 105, as indicated by line 220, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time, as indicated by the dashed line 225. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time, as indicated by the solid line 220, may be a value substantially equal to the initial rise time as indicated by the dashed line 225.

FIG. 3 is a graph illustrating additional details that may be associated with another example pressure control mode in some embodiments of the therapy system 100. In FIG. 3, the x-axis represents time and the y-axis represents negative pressure generated by the negative-pressure source 105. The target pressure in the example of FIG. 3 can vary with time in a dynamic pressure mode. For example, the target pressure may vary in the form of a triangular waveform, varying between a negative pressure of 50 and 135 mmHg with a rise time 305 set at a rate of +25 mmHg/min. and a descent time 310 set at −25 mmHg/min. In other embodiments of the therapy system 100, the triangular waveform may vary between negative pressure of 25 and 135 mmHg with a rise time 305 set at a rate of +30 mmHg/min and a descent time 310 set at −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

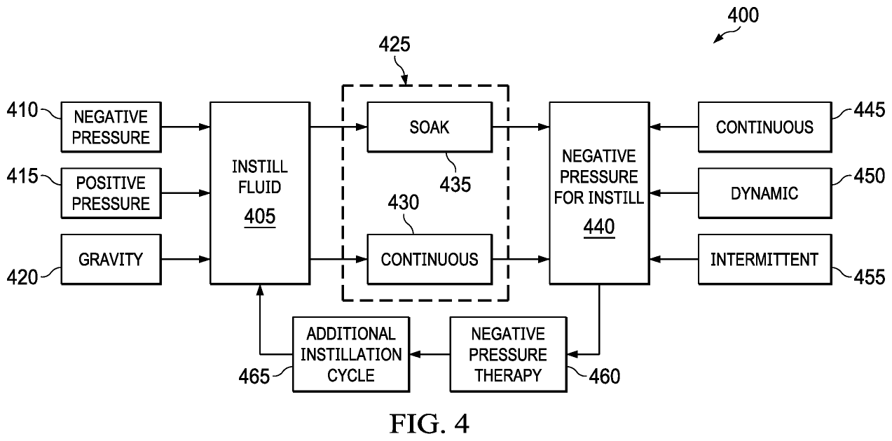

FIG. 4 is a chart illustrating details that may be associated with an example method 400 of operating the therapy system 100 to provide negative-pressure treatment and instillation treatment to the tissue interface 120. In some embodiments, the controller 130 may receive and process data, such as data related to instillation solution provided to the tissue interface 120. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 130 may also control the operation of one or more components of the therapy system 100 to instill solution, as indicated at 405. For example, the controller 130 may manage fluid distributed from the solution source 145 to the tissue interface 120. In some embodiments, fluid may be instilled to a tissue site by applying a negative pressure from the negative-pressure source 105 to reduce the pressure at the tissue site, drawing solution into the tissue interface 120, as indicated at 410. In some embodiments, solution may be instilled to a tissue site by applying a positive pressure from the positive-pressure source 150 to move solution from the solution source 145 to the tissue interface 120, as indicated at 415. Additionally or alternatively, the solution source 145 may be elevated to a height sufficient to allow gravity to move solution into the tissue interface 120, as indicated at 420.

The controller 130 may also control the fluid dynamics of instillation at 425 by providing a continuous flow of solution at 430 or an intermittent flow of solution at 435. Negative pressure may be applied to provide either continuous flow or intermittent flow of solution at 440. The application of negative pressure may be implemented to provide a continuous pressure mode of operation at 445 to achieve a continuous flow rate of instillation solution through the tissue interface 120, or it may be implemented to provide a dynamic pressure mode of operation at 450 to vary the flow rate of instillation solution through the tissue interface 120. Alternatively, the application of negative pressure may be implemented to provide an intermittent mode of operation at 455 to allow instillation solution to dwell at the tissue interface 120. In an intermittent mode, a specific fill volume and dwell time may be provided depending, for example, on the type of tissue site being treated and the type of dressing being utilized. After or during instillation of solution, negative-pressure treatment may be applied at 460. The controller 130 may be utilized to select a mode of operation and the duration of the negative pressure treatment before commencing another instillation cycle at 465 by instilling more solution at 405.

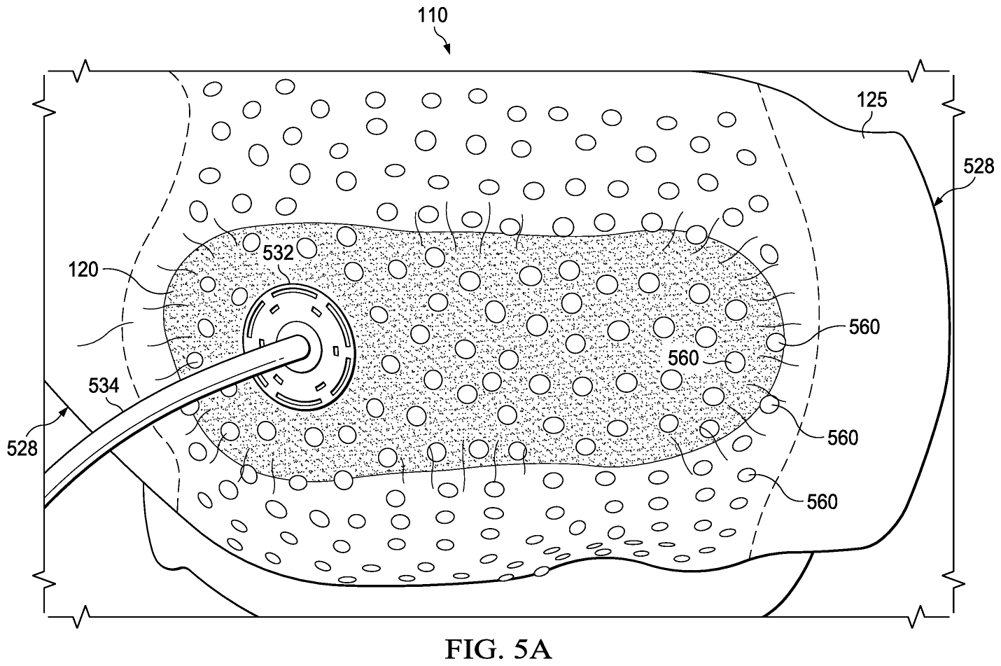

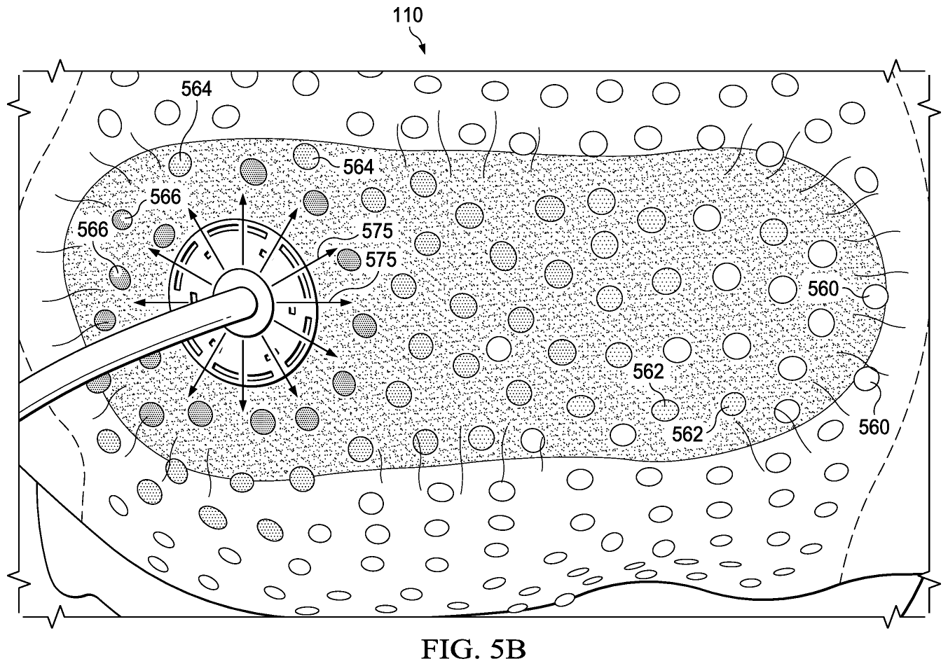

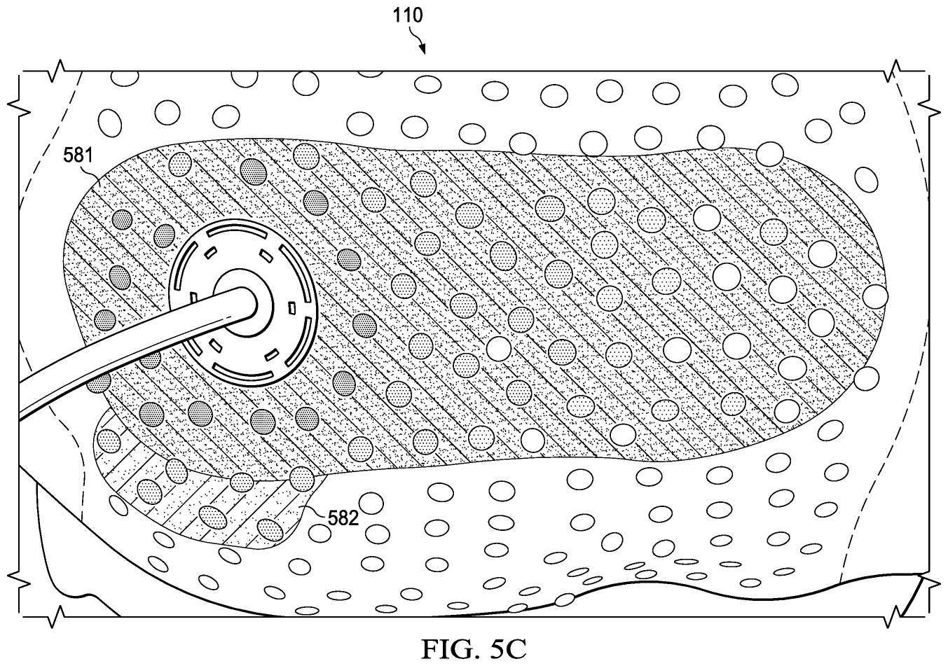

FIG. 5A is a schematic diagram illustrating additional details that may be associated with some example embodiments of the therapy system 100. In the example of FIG. 5A, the dressing 110 is applied to a wound model, and the tissue interface 120, shown here as a manifold, is sized and configured to fit on or within a tissue site (not shown but situated beneath the tissue interface 120 in FIG. 5A). The tissue interface 120 in the example of FIG. 5A includes a porous member configured as a manifold, e.g. an open-cell reticulated polyurethane foam, for instance to allow fluid instillation or fluid removal therethrough. Fluid instillation to the tissue site may be facilitated by liquid flow through an instillation interface 532, via an instillation conduit 534, for example from the positive-pressure source 150 (not shown in FIG. 5A). As applicable, fluid removal from the tissue site may occur through the same interface 532 and conduit 534 as instillation, or may alternatively occur through a separate negative-pressure interface (not shown) and negative-pressure conduit (not shown). Pressure control modes such as shown in FIG. 2-3 or therapy methods such as shown in FIG. 4 may be used to effectuate instillation, or any other appropriate mode or method of instillation may be used in conjunction with FIG. 5A. In FIG. 5A, the instillation interface 532 is shown as being disposed non-centrally with respect to the surface area of the tissue interface 120, and thus to the tissue site. However, the location of the instillation interface 532 may be chosen to be disposed or oriented anywhere over the manifolding tissue interface 532 such that instillation liquid may be provided to a sufficient portion of the tissue site.

In some embodiments, an adherent layer 528 may be disposed on at least a margin of the cover 525 and can be configured to form a seal around the tissue site.

FIG. 5A further shows a plurality of liquid contact indicators 560 disposed in a spaced array between the tissue interface 120 and the cover 125, but typically not on the margin. In some embodiments, at least a portion of the plurality of liquid contact indicators 560 can be adhered to the cover 125 on a surface facing the tissue interface 120. Additionally or alternatively, at least a portion of the plurality of liquid contact indicators 560 can be adhered to the surface of the tissue interface 120 proximate to the cover 125. In some embodiments, the adhesion of some (or each) of the liquid contact indicators 560 can be facilitated by extending the adherent layer 528 inward from the margin. Additionally or alternatively, some (or each) the liquid indicators 560 can be adhered between the tissue interface 120 and the cover 125 individually and/or using an adherent separate from the adherent layer 528. The array of liquid contact indicators 560 can advantageously be arranged to reduce, eliminate, or minimize any interference with the seal around the tissue site. In some embodiments, the liquid contact indicators 560 can be spaced from each other within the array so as to constrain wicking of the instillation liquid from one liquid contact indicator to another across the array. By controlling the spacing to constrain wicking across liquid contact indicators, false liquid contact indications can be reduced or avoided.

In some embodiments, the plurality of liquid contact indicators 560 may be a first color when not exposed to a liquid (dry) and may change to a second color, different from the first color, when exposed to a liquid such as an aqueous liquid. In some embodiments, the cover 125 can be a transparent or translucent backing layer, such as a polyurethane drape, disposed over a surface of the tissue interface 120. Optionally but preferably, the cover 125 can be transparent or translucent enough to allow an observer to visually distinguish between the first and the second color through the cover 125. In some embodiments, neither the first color nor the second color are red or pink, e.g., so that wound exudate that may be present, such as blood, would be less likely to complicate the observer's visual distinguishing between the first and second color through the cover 125. In some embodiments, the first color may include a shade of white, yellow, green, or blue, and the second color may include a shade of white, yellow, green, or blue.

In some embodiments, such as shown in the example of FIG. 5A, at least a portion of the cover 125 is not disposed over the tissue interface 120 and is also not considered a part of the margin. In such embodiments, the plurality of liquid contact indicators 560 may be disposed in this portion of the cover 125, as well as the portion of the cover 125 disposed over the tissue interface 120. Optionally but preferably, none of the plurality of liquid contact indicators 560 are disposed on or beneath the margin of the cover 125.

In some embodiments, each of the plurality of liquid contact indicators 560 may have a largest dimension from about 1 mm to about 5 mm. Nevertheless, the size or shape of the plurality of liquid contact indicators 560 may be increased or decreased to achieve desired goals, such as adequate or improved spatial resolution of the flow field. In the examples of FIGS. 5A-5C, the liquid contact indicators 560 each appear roughly circular with a diameter as the largest dimension. However, any appropriate shape or combination of shapes can be used for liquid contact indicators 560, without limitation. In some embodiments, the plurality of liquid contact indicators 560 may include two or more different sizes, at least a first size disposed directly between the cover 125 and the tissue interface 120 and at least a second size disposed between the cover 125 and the tissue surrounding the tissue site. In such different size embodiments, the first size may have a largest dimension that is larger than a largest dimension of the second size. In some embodiments, the first color, the second color, or both may be different for the plurality of liquid contact indicators 560 disposed directly between the cover 125 and the tissue interface 120 and the plurality of liquid contact indicators 560 disposed between the cover 125 and the tissue surrounding the tissue site. In some embodiments, one or both of the size and shape and one or both of the first and second colors can be different for the plurality of liquid contact indicators 560 disposed directly between the cover 125 and the tissue interface 120 and the plurality of liquid contact indicators 560 disposed between the cover 125 and the tissue surrounding the tissue site.

FIG. 5A shows a representation of the dressing 110 with all its elements fully assembled. However, it is contemplated that a dressing kit can be offered for commercial sale that may contain certain elements to be assembled to attain the dressing 110. For example, such a dressing kit could contain a tissue interface 120, comprising or consisting essentially of the manifold, and a transparent or translucent backing layer, comprising or consisting essentially of the cover 125. In addition, such a dressing kit could further contain an adherent layer, such as adherent layer 528, which may be disposed on at least a margin of the backing layer and configured to form a seal around a tissue site. In such a dressing kit, a plurality of liquid contact indicators, such as indicators 560, can be included separately, optionally with instructions to allow a user to dispose them in a spaced array between the manifold and the backing layer.

FIG. 5B is a schematic diagram illustrating additional details that may be associated with use of some example embodiments of the dressing 110 of FIG. 5A. In the example embodiment of FIG. 5B, instillation liquid flow paths 575 are shown as emanating outward from the instillation interface 532 to the tissue site (typically through the tissue interface 120 of the dressing 110). The instillation liquid can flow from the solution source 145 (not shown), e.g. as controlled via the instillation regulator (not shown), through the instillation conduit 534 to reach the instillation interface 532. FIG. 5B shows individual liquid contact indicators that have been exposed to various levels of instillation liquid, based on the instillation liquid flow paths 575. liquid contact indicators exposed to no or minimal instillation fluid are labelled 560 and tend to be furthest from the instillation interface 532 or outside a zone to which instillation flow has been directed. liquid contact indicators exposed to a relatively small amount instillation fluid are labelled 562 and tend to extend inward from the unexposed liquid contact indicators 560, typically toward the instillation interface 532. liquid contact indicators exposed to a relatively moderate amount of instillation fluid are labelled 564 and tend to extend inward from the slightly exposed liquid contact indicators 562, typically further toward the instillation interface 532. liquid contact indicators exposed to a relatively large amount instillation fluid (up to saturation level) are labelled 566 and tend to be most proximate to the instillation interface 532, typically inward from the moderately exposed liquid contact indicators 564.

In the embodiment shown in FIG. 5B, the plurality of liquid contact indicators 560 have a first color of white when dry and a second color of blue (shown in grayscale) when exposed to instillation liquid. Also in the embodiment shown in FIG. 5B, there are gradations of the second color (blue, shown in gray) on each liquid contact indicator (562, 564, 566) that are meant to indicate relative levels of exposure to instillation liquid. Although the liquid indicators are shown in FIG. 5B as exhibiting four colors representing four relative levels of instillation liquid contact, fewer and greater colors/levels are alternatively contemplated.

By mapping the pattern of instillation liquid exposure shown by the color change and its intensity, an observer can identify simultaneously both the extent of instillation liquid provision to the tissue site and the relative level of instillation liquid provision to the tissue site. For example, FIG. 5C illustrates at least these two aspects of a desirable instillation process with the dressing 110 of FIG. 5B. Often, it is desirable to have moderate to extensive instillation liquid distribution in a desired instillation flow zone 581 that essentially extends to the edges of the tissue interface 120 portion of the dressing 110. In FIG. 5C, it can be seen that a leftmost portion of the desired instillation flow zone 581 has been exposed to relatively moderate to extensive instillation liquid, by virtue of the presence of liquid contact indicators showing relatively moderate exposure 564 and of liquid contact indicators showing relatively extensive exposure 566, respectively. FIG. 5C also shows that a rightmost portion of the desired instillation flow zone 581 has been exposed to no or only relatively slight levels of instillation liquid, by virtue of the presence of liquid contact indicators showing no or minimal exposure 560 and of liquid contact indicators showing relatively slight exposure 562, respectively. This could indicate to an observer, for instance, that the instillation process was not fully complete across the tissue site. If desired, this observation could result in modification or continuation of the instillation process to a more complete level throughout the desired instillation flow zone 581.

It can often be desirable to know whether instillation liquid is being provided outside the desired instillation flow zone 581, which can be considered an instillation leak or breach in some embodiments. In FIG. 5C, the presence of liquid contact indicators showing relatively moderate exposure 564 and of liquid contact indicators showing relatively extensive exposure 566 within the instillation flow breach zone 582 indicates that such a breach has occurred. If desired, this observation could result in modification or discontinuation of the instillation process to reduce or halt instillation flow outside the desired instillation flow zone 581 that could increase the area of the instillation breach zone 582. Optionally, observations regarding growth of potential instillation flow breach zones 582 can be factored in combination with observations regarding extent of instillation flow within the desired instillation flow zone 581, and modification, continuation, or discontinuation of the instillation process can be effected based thereon.

In some embodiments, the color change of the liquid contact indicators 560 from first color to second color may be irreversible. In such embodiments, liquid indicators 566 that had experienced relatively extensive (or saturating) exposure to instillation solution from an initial instillation treatment stage of a method of operating a therapy system, e.g. as detailed in FIG. 4, would not be particularly useful in indicating further exposure to instillation solution from one or more subsequent instillation treatment stages of the therapy. Nevertheless, in such embodiments, liquid indicators 564, 562, 560 that had experienced relatively moderate, slight, or no exposure to instillation solution from an initial instillation treatment stage of a therapy could potentially indicate further exposure to, however limited and up to saturation from, instillation solution from one or more subsequent instillation treatment stages of the therapy. Additionally or alternatively in such embodiments, when assessing breach conditions, liquid indicators 564, 562, 560 from outside the desired instillation flow zone 581, which had experienced relatively moderate, slight, or no exposure to instillation solution from an initial instillation treatment stage of a therapy, could potentially still indicate further leaks into existing or additional instillation flow breach zones 582 from one or more subsequent instillation treatment stages of the therapy.

In some embodiments, the color change the color change of the liquid contact indicators 560 from first color to second color may be reversible. In such embodiments, liquid indicators 562, 564, 566 that had changed color due to experiencing some exposure to instillation solution from an initial instillation treatment stage of a therapy, e.g. as detailed in FIG. 4, could undergo steps required to regenerate their first color or to lighten the shade of second color as close to the first color as practical. In such embodiments, such at least partially regenerated liquid indicators could be useful in indicating further exposure to instillation solution from one or more subsequent instillation treatment stages of the therapy. Additionally or alternatively in such embodiments, when assessing breach conditions, such at least partially regenerated liquid indicators from outside the desired instillation flow zone 581 could potentially still indicate some or further leaks of instillation solution into existing/additional instillation flow breach zones 582 from one or more subsequent instillation treatment stages of the therapy.

In some embodiments, therapy systems for treating a tissue site with negative pressure can include a dressing, such as the dressing 110 as shown in FIGS. 5A-5C, or a dressing kit that can be assembled into such a dressing. The therapy systems may further include a fluid source, such as positive pressure source 150, coupled to and in fluid communication with the dressing and a plurality of fluid delivery pathways, such as in the tissue interface 120, that are configured to be in fluid communication with the fluid source, such as the solution source 145, and to enable fluid instillation to the tissue site.

In some embodiments, the therapy systems may further include a fluid flow conduit and a fluid connector subsystem for fluidly coupling the fluid source to the dressing for fluid instillation. In some embodiments, the therapy systems may further include a container fluidly coupled to the fluid source and the dressing and adapted to store or to provide fluid.

In some embodiments, the therapy systems may further include a negative-pressure source fluidly coupled to the dressing and configured to enable fluid removal through the dressing. In some embodiments including negative-pressure sources, the therapy systems may further include a negative-pressure conduit, a negative-pressure connector subsystem for fluidly coupling the negative-pressure source to the dressing for fluid removal, and optionally a container fluidly coupled to the negative-pressure source and the dressing and adapted to collect fluid. In some therapy system embodiments, if present, the fluid flow conduit may also function as a negative-pressure conduit. Additionally or alternatively in some therapy system embodiments, if present, the fluid connector subsystem may also function as a negative-pressure connector subsystem to fluidly couple the negative-pressure source to the dressing for fluid removal through the fluid delivery pathways, which therefore may also function as fluid removal pathways to enable fluid removal through the dressing.

In some embodiments, a dressing, an assembled dressing kit, or a therapy system containing such dressings or assembled dressing kits can be deployed in various methods for monitoring fluid distribution among a tissue site. Such methods may include deploying a fluid connector subsystem, deploying a sealing member to form a fluid seal over the tissue site, fluidly coupling the fluid connector subsystem to a fluid source, and activating the fluid source to allow fluid instillation to the tissue site. In such methods, the plurality of liquid contact indicators in their array may advantageously individually change from a first color when dry to a second color, different from the first color, when exposed to an aqueous fluid such as an instillation solution, for instance, to allow an observer to visually identify through the backing layer whether fluid instillation among the tissue site is appropriately distributed or of a proper volume. In some embodiments, a method for monitoring fluid distribution among a tissue site may include deploying a negative-pressure connector subsystem, which can be coterminous with or separate from the fluid connector subsystem, fluidly coupling the negative-pressure connector subsystem to a negative-pressure source, and activating the negative-pressure source to allow fluid removal from the tissue site.

In some embodiments, a dressing, an assembled dressing kit, or a therapy system containing such dressings or assembled dressing kits can be deployed in various methods of reducing edema for a wound (tissue site) surrounded by tissue. Such methods may include positioning the dressing, at least a portion of the system, or the dressing kit with the spaced array of liquid contact indicators over or within the wound, such that at least a portion of the adherent layer contacts the tissue surrounding the wound.

The systems, apparatuses, and methods described herein may provide significant advantages. For example, adequate instillation solution volume for a given tissue site can be a particular point of uncertainty with many users of treatment methods involving one or more instillation stages. Particularly in cases where relatively hydrophilic tissue interfaces are present in the dressing to provide a tight correlation with instillation solution flow at the tissue site, the use of a properly-spaced array of liquid contact indicators may advantageously provide a real-time visual indication of instillation flow. Such real-time visual indication of instillation flow patterns and leaks (breaches) can, in some embodiments, be used as feedback to improve or perfect instillation volume or distribution for the specific tissue site. In such situations, for instance, smartphone applications using computer vision and interpretation algorithms, such as iOn Healing™ or the like, may be used or updated to assist the use in proper tissue site instillation volume or distribution.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A dressing for treating a tissue site, the dressing comprising:
a manifold;
a backing layer disposed over a surface of the manifold, the backing layer being transparent or translucent;
an adherent layer disposed on at least a margin of the backing layer and configured to form a seal around the tissue site; and
a plurality of liquid contact indicators disposed in a spaced array between the manifold and the backing layer.

2. The dressing of claim 1, wherein at least a portion of the plurality of liquid contact indicators are adhered to the backing layer on a surface facing the manifold.

3. The dressing of claim 1 or claim 2, wherein at least a portion of the plurality of liquid contact indicators are adhered to the surface of the manifold proximate to the backing layer.

4. The dressing of any one of claims 1-3, wherein the plurality of liquid contact indicators are a first color when dry and change to a second color, different from the first color, when exposed to an aqueous fluid, and wherein the backing layer is transparent or translucent enough to allow an observer to visually distinguish between the first and the second color through the backing layer.

5. The dressing of claim 4, wherein neither the first color nor the second color is red or pink.

6. The dressing of claim 4 or claim 5, wherein the first color comprises white, yellow, green, or blue, and the second color comprises white, yellow, green, or blue.

7. The dressing of any one of claims 1-6, wherein each of the plurality of liquid contact indicators has a largest dimension from about 1 mm to about 5 mm.

8. The dressing of any one of claims 1-7, wherein at least a portion of the backing layer is not disposed over the manifold and is also not considered a part of the margin.

9. The dressing of claim 8, wherein the plurality of liquid contact indicators are not disposed on the margin of the backing layer.

10. The dressing of any one of claims 1-9, wherein the plurality of liquid contact indicators comprises two or more different sizes, at least a first size disposed directly between the backing layer and the manifold and at least a second size configured to be disposed between the backing layer and tissue surrounding the tissue site.

11. The dressing of claim 10, wherein the first size has a largest dimension that is larger than a largest dimension of the second size.

12. The dressing of any one of claims 1-11, wherein the manifold comprises open-cell reticulated polyurethane foam.

13. The dressing of any one of claims 1-12, wherein the backing layer comprises a polyurethane drape.

14. A dressing kit comprising:
a dressing for treating a tissue site, the dressing comprising:
a manifold;
a backing layer disposed over a surface of the manifold, the backing layer being transparent or translucent; and
an adherent layer disposed on at least a margin of the backing layer and configured to form a seal around the tissue site; and
a plurality of liquid contact indicators configured to be disposed in a spaced array between the manifold and the backing layer.

15. The dressing kit of claim 14, wherein each of the plurality of liquid contact indicators comprises an adhesive layer configured to be adhered to the backing layer or to the manifold, as well as a liquid contact indicator layer that is a first color when dry and that changes to a second color, different from the first color, when exposed to an aqueous fluid, and wherein the backing layer is transparent or translucent enough to allow an observer to visually distinguish between the first and the second color through the backing layer.

16. A system for treating a tissue site with negative pressure, the system comprising:
a dressing according to any one of claims 1-13 or a dressing kit according to any one of claims 14-15;
a fluid source coupled to and in fluid communication with the dressing; and
a plurality of fluid delivery pathways configured to be in fluid communication with the fluid source and to enable fluid instillation to the tissue site.

17. The system of claim 16, further comprising:
a fluid flow conduit; and
a fluid connector subsystem for fluidly coupling the fluid source to the dressing for fluid instillation.

18. The system of claim 17, further comprising a container fluidly coupled to the fluid source and the dressing and adapted to store or to provide fluid.

19. The system of any one of claims 16-18, further comprising a negative-pressure source fluidly coupled to the dressing and configured to enable fluid removal through the dressing.

20. The system of claim 19, further comprising:
a negative-pressure conduit;
a negative-pressure connector subsystem for fluidly coupling the negative-pressure source to the dressing for fluid removal; and
optionally a container fluidly coupled to the negative-pressure source and the dressing and adapted to collect fluid.

21. The system of claim 19, wherein:
if present, the fluid flow conduit also functions as a negative-pressure conduit; and
if present, the fluid connector subsystem also functions as a negative-pressure connector subsystem to fluidly couple the negative-pressure source to the dressing for fluid removal through the fluid delivery pathways that therefore also function as fluid removal pathways to enable fluid removal through the dressing.

22. A method for monitoring fluid distribution among a tissue site, the method comprising:
deploying on or within the tissue site a dressing according to any one of claims 1-13, at least a portion of a system for treating a tissue site according to any one of claims 16-21, or a dressing kit according to any one of claims 14-15 after the plurality of liquid contact indicators have been disposed in a spaced array between the manifold and the backing layer;
deploying a fluid connector subsystem;
deploying a sealing member to form a fluid seal over the tissue site;
fluidly coupling the fluid connector subsystem to a fluid source; and
activating the fluid source to allow fluid instillation to the tissue site,
wherein the plurality of liquid contact indicators in their array individually changing from a first color when dry to a second color, different from the first color, when exposed to an aqueous fluid allows an observer to visually identify through the backing layer whether fluid instillation among the tissue site is appropriately distributed or of a proper volume.

23. The method of claim 22, wherein a portion of the plurality of liquid contact indicators disposed directly between the backing layer and the manifold allow the observer to visually identify through the backing layer whether fluid is appropriately distributed across the tissue site or across only a portion of the tissue site.

24. The method of claim 22 or claim 23, wherein a portion of the plurality of liquid contact indicators disposed between the backing layer and the tissue surrounding the tissue site allow the observer to visually identify through the backing layer whether fluid has dispersed past a boundary of the tissue site to the tissue surrounding the tissue site.

25. The method of any one of claims 22-24, wherein the tissue site comprises a peritoneal or abdominal cavity, a burn, a graft, an overhang wound, a post-operative wound, a puncture or a fistula.

26. The method of any one of claims 22-25, further comprising:
deploying a negative-pressure connector subsystem, which can be coterminous with or separate from the fluid connector subsystem;
fluidly coupling the negative-pressure connector subsystem to a negative-pressure source; and
activating the negative-pressure source to allow fluid removal from the tissue site.

27. A method of reducing edema for a wound surrounded by tissue, the method comprising:
providing a dressing according to any one of claims 1-13, at least a portion of a system for treating a tissue site according to any one of claims 16-21, or a dressing kit according to any one of claims 14-15 after the plurality of liquid contact indicators have been disposed in a spaced array between the manifold and the backing layer; and
positioning the dressing, at least a portion of the system, or the dressing kit with the spaced array of liquid contact indicators over or within the wound, such that at least a portion of the adherent layer contacts the tissue surrounding the wound.
